(19) United States
(12) Patent Application Publication
(10) Pub. No.: US 2014/0001028 A1
(43) Pub. Date: Jan. 2, 2014

(54) METHOD FOR CONVERTING METHANE INTO OXYGENATED DERIVATIVES OF ONE CARBON ATOM

(71) Applicants: Francesc Sastre Calabuig, Rotova (Valencia) (ES); Avelino Corma Canos, Valencia (ES); Hermenegildo García Gómez, Sueca (Valencia) (ES)

(72) Inventors: Francesc Sastre Calabuig, Rotova (Valencia) (ES); Avelino Corma Canos, Valencia (ES); Hermenegildo García Gómez, Sueca (Valencia) (ES)

(21) Appl. No.: 13/981,691

(22) PCT Filed: Jan. 10, 2012
(86) PCT No.: PCT/ES2012/070008

§ 371 Date: Sep. 17, 2013

(30) Foreign Application Priority Data
Jan. 26, 2011 (ES) P201130088

(51) Int. Cl. C07C 29/36

(52) U.S. Cl. CPC C07C 29/36
USPC 204/157.63

(57) ABSTRACT

Method for converting methane into oxygenated derivates, characterized in that it comprises irradiating a solid surface using UV light with a wavelength of less than 200 nm and in that it takes place on the surface of a solid, preferably a microporous solid, wherein the selectivities achieved are greater than 90% for conversions of about 10%.

### FIELD OF THE INVENTION

The present invention relates to a method for converting methane into derivatives of one carbon atom containing one or more oxygen atoms by using ultraviolet light with a wavelength of less than 200 nm in the presence of a solid material.

### STATE OF THE ART

A main component of natural gas is methane which is a hydrocarbon that has the lowest boiling point and is difficult to liquefy. It would be expedient to be able to convert methane into liquid derivatives, which would facilitate the transport and the use of this fuel from the places where the natural gas mining fields are located to the locations where it is consumed.

Currently, the main industrial process for converting methane consists on vapor reforming (equation 1) where, at high temperatures, the methane reacts with water vapor to produce carbon monoxide and hydrogen (J. N. Armor, Applied Catalysis a-General 1999, 176, 159; J. P. Vanhook, Catalysis Reviews-Science and Engineering 1980, 21, 1.).

The gas resulting from the reforming of the methane may be referred to as synthesis gas and it is susceptible to further conversion and, in particular, to the separation of its components or the reduction of the CO content by means of processes such as water gas shift. However, the industrial process of vapor reforming is carried out in large facilities which operate at optimal conditions and which require large investments of capital as well as sufficiently large areas of land to install the chemical plant. Developing processes for converting methane into easily transportable liquid derivatives, which can be carried out in small facilities, and which require low capital investments, would have a significant economic impact.

In this connection, studies are currently being conducted examining the direct conversion of methane into aromatic hydrocarbons, oxidative coupling to ethylene as well as the direct oxidation to methanol. Scheme 1 summarizes some of the processes being studied in order to convert methane into other derivatives in an alternative manner to vapor reforming.

Vapor-reforming CH₄+H₂O→CO+3H₂ Equation 1

Aromatization CH₄→C₆H₆ Equation 2

Oxidative coupling 2CH₄+O₂→CH₂=CH₂+H₂O Equation 3

Oxidation to methanol CH₄+½O₂→CH₃OH Equation 4

However, all of the processes for converting methane are limited by the high temperatures required and by the fact that the catalysts are subject to deactivation over time.

A general process allowing methane to be converted is based on attacking hydroxyl radicals (OH) on this compound. Due to the greater stability of the O—H bond, the hydroxyl radical is able to abstract a hydrogen atom from the methane to produce the methyl radical. This methyl radical, which acts as a primary specie, may undergo coupling to produce ethane or methanol according to its reaction with another methyl radical or a hydroxyl radical, respectively. The equations 4 to 9 summarize the processes which may be derived from attacking the hydroxyl radical on the methane.

OH^{•}+CH₄→H₂O+CH₃^{•} Equation 5

CH₃^{•}+CH₃^{•}→CH₃CH₃ Equation 6

OH^{•}+CH₃CH₃→H₂O+CH₃CH₃^{•} Equation 7

CH₃^{•}+CH₃CH₂^{·•}→CH₃CH₂CH₃ Equation 8

CH₃^{•}+O₂→CH₃O—O^{•} Equation 9

CH₃O—O^{•}+CH₄→CH₃O'OH+CH₃^{•} Equation 10

Although the activation of methane via radicals and in particular by using the hydroxyl radical is a general process for the functionalization of this hydrocarbon and, in addition, it may be carried out at moderate temperatures, these reactions occur with a very low selectivity even for low conversions. The reason for this low selectivity is that the primary reaction products undergo free-radical attack much more quickly than methane and consequently these primary products are converted to secondary products, thus obtaining complex reaction mixtures which are difficult to handle and have a low economic value. The attempts to obtain selectivity in this free-radical process, both in the gaseous and liquid phases, have not produced favorable results which is why this type of mechanism has generally been discarded when converting methane.

The hydroxyl radical which may be used to activate the methane may be obtained, mainly, from hydrogen peroxide by means of reduction with an iron salt (II) or another transition metal (Fenton's reaction), however, this process requires aqueous medium wherein the solubility of the methane is very low.

Alternatively, the hydroxyl radical may be photocatalytically generated such as for example by means of irradiation of anatase-phase titanium dioxide using ultraviolet light with a wavelength of less than 350 nm. Finally, and in direct connection with the present invention, the hydroxyl radical may be generated by means of homolytic cleavage of the hydrogen-oxygen bond of the water caused by light with a short wavelength, lower than 200 nm. The water exhibits no absorption in the visible ultraviolet spectrum in the region between 200 and 800 nm which is why the direct irradiation of the latter requires wavelengths of less than 190 nm. The water molecule may be excited directly using a mercury lamp at low pressure, which emits at 185 nm, while it has been described that this molecule undergoes homolytic cleavage of the hydrogen-oxygen bond with a quantum yield close to one when it is irradiated at 185 nm (N. Getoff, G. O. Schenk, Photochem. Photobiol. 1968, 8, 167.).

### DESCRIPTION OF THE INVENTION

The present invention relates to a method for converting methane into oxygenated derivates with one carbon atom by means of irradiation with ultraviolet light with a wavelength of less than 200 nm in the presence of a solid material. It is thereby possible to achieve selectivity in the conversion reactions into a methane derivative, with the reaction being carried out on a surface or confined space.

The foundation of the present invention is that the irradiation of the hydroxyl groups present on the surface of many materials, with deep ultraviolet light, gives rise to oxyl radicals (—O^{·}), as it is indicated in equation 11 in the case of irradiating a silanol group.

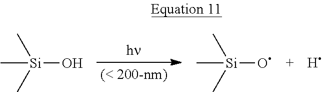

These oxyl radicals are able to abstract a hydrogen atom from the methane to produce methyl radicals which remain absorbed on the surface, and they may even form covalent bonds with superficial groups. These methyl radicals may also become trapped by molecular oxygen (equation 9) if molecular oxygen is present in the reaction medium, producing alkyl peroxide radicals which also remain adsorbed or even covalently bonded to the surface of the solid or in the interior of the micropores of the material. The reason of the selectivity achieved in the process is derived of confining the free-radical reaction on a surface or inside a restricted space, thus minimizing the secondary reactions.

The method of the present invention is based on using solid materials which have a high density of superficial hydroxyl groups, which also have suitable acid-base properties. Porous materials are among the preferred materials of the invention, particularly those with a pore size less than 2 nm and preferably in the interval of the micropores.

Some types of materials wherein high levels of selectivity are achieved when converting methane by using strong ultraviolet light are zeolites, both with a large pore size and a medium pore size. In this case, the crystalline structure of the material defines channels and cavities in which the hydroxyl groups are located and where the conversion of methane by means of irradiation takes place.

Deuterium gas lamps (higher emission intensity at 165 nm) and low-pressure mercury vapor lamps (185 nm) with suitable power and preferably with few watts may be used to carry out the irradiation.

The irradiation stage may be carried out in a container with a window made of Suprasil quartz or halides of alkali earth metal ions (MgF₂) through which it is illuminated using the lamp and which contains a thin layer of solid material where the reaction will take place, the latter being in contact with an atmosphere containing methane and which may also contain other gases such as nitrogen. Nitrogen or another inert gas may be used as the standard, enabling the quantification of the converted methane and the products formed and the material balances. Moreover, if oxygen is present in the medium, it may trap the radicals which escape the gaseous phase. These gases may be at atmospheric pressure, below atmospheric pressure or above atmospheric pressure, as required.

By using isotopically labeled methane such that it is enriched with ¹³C and by using ¹³C Nuclear Magnetic Resonance (NMR) for solids, the methane reaction may be established within the material and the products formed on the external or internal surface of the solid may be characterized and quantified.

### Use of Preferred Materials

There are a number of types of aluminosilicates suitable for the conversion of methane described in this document, however, this does not limit the present invention as it is not restricted to the following materials.

The following are among the preferred materials of this invention:
a zeolite beta without aluminum and prepared using tetramethylammonium hydroxide in the synthesis gel preparation.
a medium pore size zeolite beta.
a mesoporous or microporous aluminosilicate.
a mesoporous whose structure is of the MCM-41 type.
an amorphous siliceous material formed by dense nanoparticles without micropores.
a mesoporous or microporous aluminosilicate formed by metallic oxide nanoparticles such as CeO₂, TiO₂, Al₂O₃ and ZrO₂ or combinations thereof in any of their phases and being structured or unstructured.
Mixtures of the materials mentioned above.

### BRIEF DESCRIPTION OF THE FIGURES

FIG. 1 shows the reaction products determined using ¹³C solid-state

NMR that are present in zeolite beta with a Si to Al ratio of 22 and prepared using the tetramethylammonium hydroxide method described in the literature (M. A. Camblor, A. Corma, S, Valencia, J. Mat. Chem. 1998, 8, 2127).

FIG. 2 shows ¹³C NMR spectra obtained when irradiating methane isotopically labeled with ¹³C during each of the stages of several cycles of irradiation/extraction as well as the spectrum obtained from extraction water. This figure shows how it is possible to recover the oxygenated derivatives formed from the material (b and e spectra) and to reuse the same solid in successive cycles.

FIG. 3 shows a ¹³C NMR spectrum using the cross polarization technique on a sample of zeolite beta subjected to irradiation.

FIG. 4 shows a superposition of ¹³C NMR spectra corresponding to each of the stages of irradiation/extraction and reuse carried out according to Example 3.

### DETAILED DESCRIPTION OF THE INVENTION

The present invention relates to a method for converting methane characterized in that it comprises: irradiating a solid with wavelengths equal to or less than 200 nm and producing a free-radical reaction in a photochemical reactor, forming oxygenated compounds of one carbon atom on the surface of said solid.

The photochemical reactor may have a deuterium lamp and a magnesium fluoride window or a mercury lamp and a quartz window.

The solid used may be a mesoporous or microporous aluminosilicate.

The solid used may, for example, be a medium or small pore size zeolite. Said zeolite may be a zeolite beta with a silicon to aluminum ratio between 10 and infinity.

The method may be carried out by means of one cycle having two stages:
the first stage comprises the photochemical irradiation, in which the methane derivatives are formed both in the gaseous phase and, preferably, inside the solid, and
the second stage comprises the desorption of the oxygenate compounds present in the solid.

The second stage may be carried out by means of washing the material with water at 150° C.

The method may also be carried out continuously, introducing a gas flow into the photo-reactor which includes methane and oxygen, wherein the exit gases contain methanol and other oxygenated derivatives. The temperature is preferably 80° C.

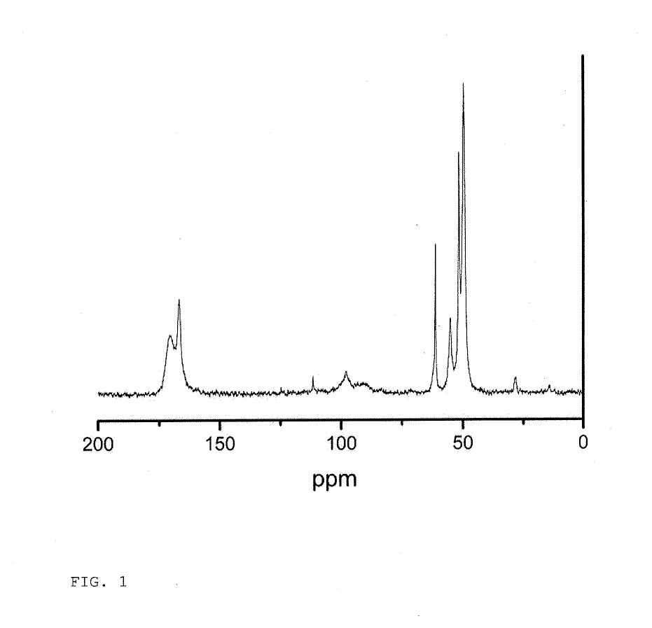

FIG. 1 shows a solid-state ¹³C NMR spectrum of a zeolite beta (Si to Al 22), which has been subjected to irradiation at 165 nm in an atmosphere containing methane and air.

As it may be seen from said FIG. 1, the signals appearing between 45 and 60 ppm correspond to adsorbed methanol and methoxy groups fixed to different silanol groups. Furthermore, the signals between 80 and 100 ppm correspond to hemi- and acetal compounds derived from formaldehyde and the signals around 160 ppm correspond to formic acid present in the material. The proportion between these three types of oxygenated species derived from methane depends on the nature of the solid used for the irradiation which also affects the conversion of methane achieved. These data, the variation of the product distribution and the methane conversion as a function of the nature of the solid, indicate that the reaction takes place on the external and internal surface of the material. Moreover, control tests, in which methane is irradiated in the gaseous phase in the presence of water vapor, do not lead to reaction products being observed in the majority of the conditions applied.

Methane converted using photochemical reaction with deep ultraviolet light on solid surfaces may be determined using gas chromatography by comparing the relative area of methane and nitrogen before and after the irradiation. In addition, gas chromatography using TCD and FID detectors allows the presence of products derived from methane to be detected in the gaseous phase, the most significant of these products including hydrogen, ethane and methanol. Table 1 shows the product distribution determined in the gaseous phase for the irradiation of methane with light at 165 nm on different supports.

TABLE 1
Composition data of the gaseous phase in contact with the solid
after irradiating a mixture of methane and air with UV light at 165 nm.

| | | | | Beta | Beta | | |
|---|---|---|---|---|---|---|---|
| | | Beta | Beta | (Si, | (Al, | Silica | Beta |
| Selectivity | Silicalite | (Si, F) | (Al, F) | OH) | OH) | gel | 811 |
| Ethane | 30.81 | 44.56 | 38.22 | 44.77 | 46.02 | 25.80 | 29.20 |
| Propane | 2.25 | 3.28 | 2.64 | 6.01 | 7.10 | 13.81 | 5.70 |
| Methanol | 17.26 | 20.84 | 12.34 | 14.28 | 15.50 | 13.11 | 7.60 |
| Hydrogen | 21.43 | 21.81 | 16.05 | 8.52 | 16.33 | 26.34 | 7.70 |

The zeolite used in the present invention may be hydrated, however the level of hydration does not affect its activity. Moreover, zeolites which have different structures and composition may be used. In addition, other meso, micro or macroporous solids as well as dense materials without pores may also be used. The conversion of methane may be carried out on all types of silica, alumina and oxides of various metals, this list not being exhaustive.

The irradiation may be carried out for periods between five minutes and many hundreds of hours, producing a conversion increase and a variation of the product distribution. The most notable aspect is that methane conversions of about 10% with a global selectivity towards oxygenated derivatives of one carbon atom greater than 90% may be obtained, by optimizing irradiation time, the nature of the material and the quantity of oxygen present. This result is surprising and could not be deduced from the state of the art as with this level of conversion in a free-radical reaction, the selectivity towards oxygenated compounds in the gaseous phase is much lower and of little use.

The conversion process of methane into oxygenated derivatives with one carbon atom may be carried out by means of one cycle in two stages as explained above.

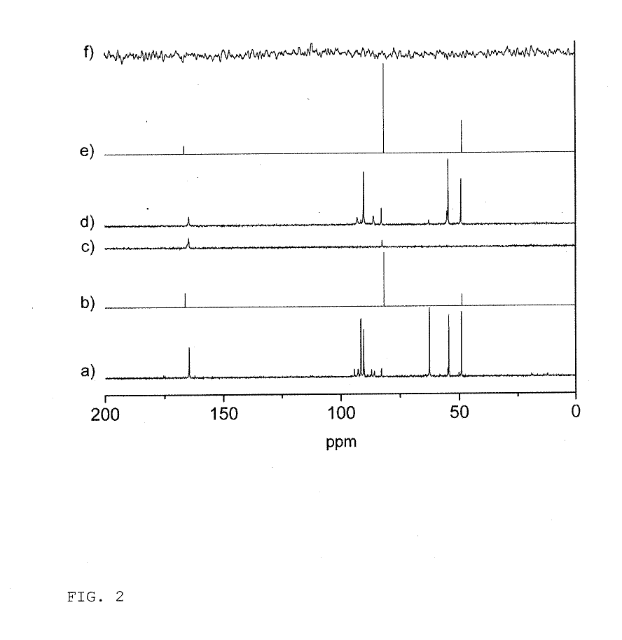

FIG. 2 shows the solid-state ¹³C NMR spectra (a, c, d and f) or in aqueous dissolution (spectra b and e) obtained after irradiating a zeolite beta (Si to Al 22) at 165 nm in contact with a gaseous phase containing methane (80%) and air (20%) at a total pressure of 0.5 bar. Spectrum a corresponds to the spectrum of the zeolite after irradiation. Spectrum b corresponds to the water extraction of the zeolite once it has been irradiated. Spectrum c corresponds to the zeolite beta, which was irradiated after being extracted with water. The d, e and f spectra correspond to a second irradiation/extraction cycle.

Alternatively it is possible to use a process involving only one stage carried out continuously in which a flow containing a mixture of methane, nitrogen and oxygen or other gases containing methane enters the photochemical reactor and undergoes conversion on the surface of the material while the products in the gaseous phase are removed from the reactor. This continuous process may be carried out at temperatures above room temperature and preferably between 40° C. and 100° C. such that it favors the desorption of the products from the solid to the gaseous phase, achieving a stationary mixture of oxygenates in the gaseous phase.

### EXAMPLES

Non-limiting examples of the present invention will be described below.

### Example 1

### Nuclear Magnetic Resonance Analysis of the Reaction Products using Methane Isotopically Enriched with ¹³C

A zeolite beta tablet with a mass of 100 mg is placed in the interior of a photochemical cylindrical reactor measuring 55 mm in diameter and 61 mm in length, provided with a magnesium fluoride window and having attached a deuterium lamp in contact with the window and whose nominal power is 1.18 W. This zeolite is obtained following the method described in the literature (A. Corma, M. T. Navarro, F. Rey, S. Valencia, Chemical Communications 2001, 1486.) and is characterized by a surface area of 490 m²/g and by containing no aluminum in the network. Before introducing the pulverant material into the photo-reactor, it is compressed in a hydraulic press at a pressure of 1 torr/cm² for 1 minute, thereby obtaining a disc with a diameter of 10 mm. After introducing the solid, the photo-reactor is charged with 99.9% pure methane, enriched with 99% ¹³C up to a pressure of 0.8 bar. The photo-reactor is then charged with air up to a pressure of 1.2 bar. The lamp is switched on and the irradiation takes place for a period of 1 h. The irradiation is carried out at room temperature (25° C.). After this time has passed, the gaseous phase is analyzed injecting an aliquot part of the gas present in the foto-reactor using a syringe into a gas chromatograph HP5890 which has a system of two columns in series and argon as the carrier gas. Hydrogen, nitrogen and methane are separated into a molecular sieve column 15 m in length and with an internal diameter of 0.53 mm connected to a thermal conductivity detector. The remainder components are separated in an alumina plot column 50 m in length and with an internal diameter of 0.5 mm which is connected to a flame ionization detector (FID). The composition and percentage of each component in the gaseous phase is determined by comparing with standards and by means of the response factor.

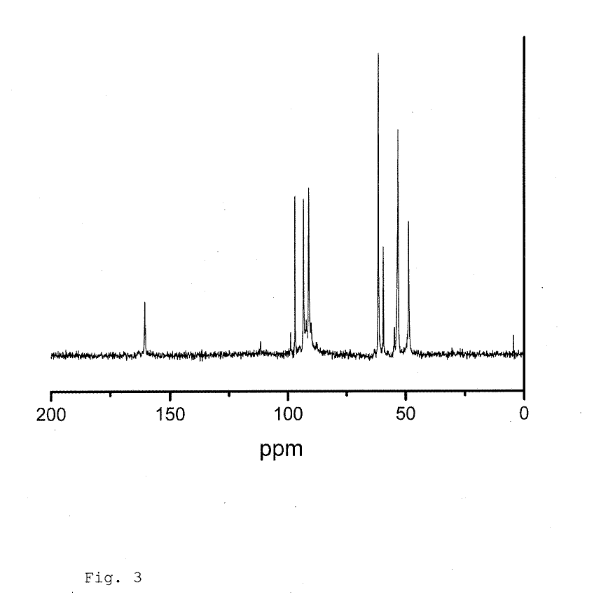

In addition, the zeolite disc is ground and a part thereof is subjected to elemental combustion analysis to determine its carbon content. The remainder sample of zeolite beta subjected to irradiation is analyzed by ¹³C NMR using the cross polarization technique, thereby obtaining the spectrum shown in FIG. 3. The methane conversion data is obtained by comparing the initial moles introduced into the photo-reactor with the methane moles present at the end of the irradiation process by using the nitrogen chromatographic peak as reference. The composition of the organic material present in the zeolite is obtained by combining the elemental analysis data with the ¹³C NMR spectrum.

FIG. 3 shows the recorded solid-state ¹³C NMR in a sample of zeolite beta which has been subjected to irradiation using a deuterium lamp for 1 h in contact with a methane and air atmosphere.

### Example 2

### Converting Ultra-Pure Methane by Photochemical Radiation in the Presence of Pure Silica Zeolite Beta

This example uses the same experimental method as example 1, however, it uses high-purity commercial methane (99.9995%) as starting material. In this case, the conversion and the quantity of organic material present in the zeolite is determined as in example 1, however, a direct analysis of the products present in the solid by ¹³C NMR is not possible. Nevertheless, the material absorbed into the solid is desorbed by treating the solid in an autoclave containing 2 ml of water which is heated to 150° C. for 1 h. The water from the autoclave is recovered, separated from the solid and analyzed by liquid ¹³C NMR, observing a product distribution analogous to that described in example 1.

### Example 3

### Methane Conversion Process in Two Stages wherein the Irradiation Step is Followed by Extraction of the Organic Components in the Solid and Reuse of the Zeolite

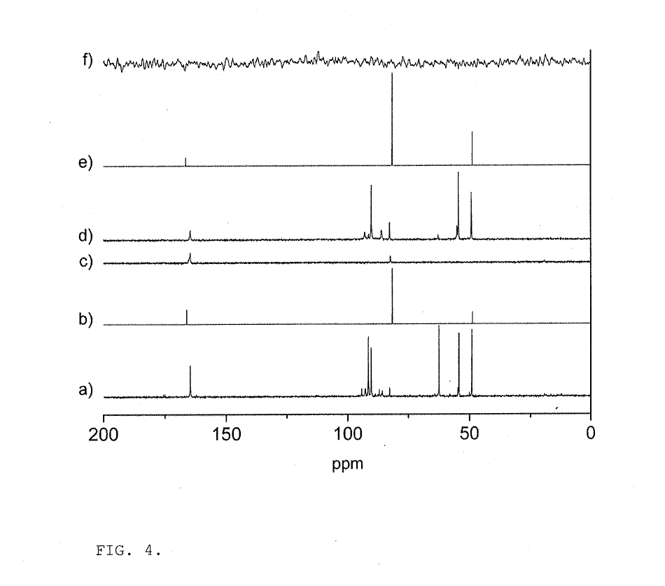

The same procedure is followed for this example as is described in example 1 using methane enriched with ¹³C and subjected to irradiation for 1 h. After this time period and after recording the solid-state ¹³C NMR spectrum, the second stage is carried out, in which the ground zeolite is placed in the autoclave and subjected to extraction with water (2 ml) at 150° C. for 1 h. The ¹³C NMR spectrum of the solid is again recorded, observing the complete disappearance of the signals corresponding to the oxygenated products derived from the methane. At the same time, ¹³C NMR of the extraction H₂O indicates the presence of methanol, formaldehyde and formic acid in a proportion of 34%, 60.6% and 5.4%, thus demonstrating that the extraction process was successful. In addition, the zeolite recovered following the autoclave, in which the photochemical reaction products were desorbed, is used in a second cycle of irradiation/extraction, achieving the same results in terms of conversion and product distribution as those obtained in the first cycle. FIG. 4 shows a superposition of the ¹³C NMR spectra corresponding to each of the stages of irradiation/extraction and reuse.

FIG. 4. ¹³C NMR spectra recorded in solid state (a, c, d and f) and liquid state (b and e) for a zeolite beta after being irradiated in contact with an atmosphere of methane and air (20%) at 1 bar for 1 h using a deuterium lamp (a). Sample a was subjected to extraction with water; the spectra of extracted water (spectrum b) and those of the zeolite following the extraction (c) were recorded. Spectra d, e and f correspond to a second cycle of irradiation/extraction.

### Example 4

### Continuous Irradiation at a Temperature of 80° C.

This example is carried out using a photochemical reactor with a deuterium lamp which emits at 165 nm and a magnesium fluoride window, whose body allows a flow of gas to enter and exit. Zeolite beta identical to the sample in example 1 is placed inside the reactor. The reactor is fed with a mixture of methane, nitrogen and oxygen in a proportion of 60%, 30% and 10% and the flow of the reactor is 0.5 ml/min. The body of the reactor is heated by electrical resistance and the temperature of the latter is regulated to 80° C. by means of a thermocouple. The outlet of the reactor has a valve allowing the pressure of the reactor to be constantly maintained at 0.5 bar. The resulting effluent is analyzed directly in a gas chromatograph. When the stationary state is reached, methane conversions about 3% are observed and in addition to hydrogen and smaller quantities of ethane, 2% methanol is also present.

### Example 5

### Use of a Photo-Reactor with a Mercury Lamp

The experimental process and the analysis methods in this example are identical to those described in example 1, with the difference that the lamp used is not a deuterium lamp, but rather a mercury-vapor lamp at low pressure. Said lamp has a maximum emission intensity of 185 nm and an output at this wavelength of 4 W. Moreover, the body of this reactor is a cylinder made of Suprasil quartz with a diameter of 60 mm and a length of 142 mm, wherein the solid material is provided as a fine bed of zeolite particles previously pressed, ground and sieved between 400 and 200 mesh. Due to the difference in wavelength, absorptivity of the hydroxyl groups and light output, the irradiation times necessary when using this reactor are considerably lower, enabling conversions such as those indicated in example 1 to be achieved by irradiating for only 5 min.

1. Method for converting methane, wherein it comprises: irradiating a solid with wavelengths equal to or less than 200 nm and producing a free-radical reaction in a photochemical reactor, forming oxygenated compounds of one carbon atom on the surface of said solid.

2. Method according to claim 1, wherein the photochemical reactor has a deuterium lamp and a magnesium fluoride window.

3. Method according to claim 1, wherein the photochemical reactor has a mercury lamp and a quartz window.

4. Method according to claim 1, wherein the solid used is a mesoporous or microporous aluminosilicate.

5. Method according to claim 4, wherein the solid used is a medium or small pore size zeolite.

6. Method according to claim 5, wherein the zeolite is a zeolite beta with a silicon to aluminum ratio between 10 and infinity.

7. Method according to claim 1, wherein it may be carried out by means of one cycle in two stages:
the first stage comprises the photochemical irradiation, in which the methane derivatives are formed both in the gaseous phase and, preferably, inside the solid, and
the second stage comprises the desorption of the oxygenate compounds present in the solid.

8. Method according to claim 7, wherein the second stage is carried out by washing the material with water at 150° C.

9. A method according to claim 1, wherein it is carried out continuously by introducing into the photo-reactor a gas flow, which includes methane and oxygen and wherein the exit gases contain methanol and other oxygenated derivatives.

10. A method according to claim 9, wherein it is carried out at a temperature of 80° C.

11. Use of a zeolite beta selected from:
a zeolite beta without aluminum and prepared using tetramethylammonium hydroxide in the synthesis gel preparation,
a medium pore size zeolite beta,
in a method as defined in claim 1.

12. Use of a mesoporous or microporous aluminosilicate in a method as the one defined in claim 1.

13. Use according to claim 12, wherein the mesoporous aluminosilicate has a MCM-41 type structure.

14. Use of an amorphous siliceous material formed by dense nanoparticles without micropores in a method as defined in claim 1.

15. Use of a mesoporous or microporous aluminosilicate formed by metallic oxide nanoparticles in a method as the one defined in claim 1.

16. Use according to claim 15, wherein the metallic oxide is selected from CeO₂, TiO₂, Al₂O₃ and ZrO₂ or combinations thereof, in any of their phases and being structured or unstructured.
